(19) United States
(12) Patent Application Publication
(10) Pub. No.: US 2017/0232052 A1
(43) Pub. Date: Aug. 17, 2017

(54) LOWERING IRON

(71) Applicant: Kim A. Anderson, Merced, CA (US)

(72) Inventor: Kim A. Anderson, Merced, CA (US)

(21) Appl. No.: 15/041,627

(22) Filed: Feb. 11, 2016

(51) Int. Cl. A61K 36/68; G01N 33/84; A61K 36/23; A61K 9/20; A61K 36/63; A61K 36/734

(52) U.S. Cl. CPC A61K 36/68; A61K 36/63; A61K 36/734; A61K 36/23; A61K 9/20; A23L 1/3002; A23L 1/293; G01N 33/84; A23V 2002/00; G01N 2800/52; G01N 2800/22

(57) ABSTRACT

Lower iron in blood can be accomplished by checking iron level in blood and taking daily an amount of Psyllium husk predetermined to be effective in reducing iron level in blood. Iron level is monitored. Daily intake of Psyllium husk is stopped or significantly reduced when iron level in blood is detected to be at a desired level.

### BACKGROUND

Iron in blood is used to carry oxygen on hemoglobin in blood. Iron does this by losing or gaining one electron going from the ferrous (Fe++) to the ferric (Fe+++) state. Iron is also a potent pro-oxidant, catalyzing the production of free radicals that can destroy cells and tissue. As red cells break down over time, the iron in the hemoglobin is released. Excess iron can be stored in body tissues and thus be detrimental to health.

Iron overload is typically treated with phlebotomy or with iron chelation therapy. Phlebotomy is the process of taking blood out of the body. As blood is removed, iron is removed as well. If anemia (low red cell count) is present in addition to iron overload, phlebotomy may not be a viable option. Phlebotomy can require several weeks of blood removal to be effective.

Iron chelation is a drug therapy that uses drugs called iron chelators to remove extra iron. Side effects can include gastrointestinal disturbances, skin rashes and liver function failure.

### DESCRIPTION OF THE EMBODIMENT

Iron overload in blood can be reduced by use of a regime that includes daily intake of Psyllium husk. Psyllium is a soluble fiber, which is a seed husk that comes from plants.

For example, 500 milligram of Psyllium husk can be taken three times per day in a regime that avoids intake of iron. The regime may include, for example: (1) drinking lots of fluids, particularly water or juice; (2) eating only foods that are low in iron or have no iron; (3) avoiding blood transfusions; (4) No intake of iron supplements such as ferrous sulfide; (5) intake of three meals per day; (6) monitoring of iron counts in blood.

Dosage amounts of Psyllium husk can be varied based on a number of factors including weight of patient, age of patient, and optimization based on experimentation.

For example, Psyllium husk can be integrated with other ingredients into a pill form. For example, 500 milligrams of Psyllium husk can be combined with 60 milligrams of Olive leaf; 40 milligrams of Hawthore berry and 400 milligrams of parsley to produce a pill to be taken conveniently. Different amounts of each ingredient can be used. Also different ingredients can be used. For iron removal, the active ingredient is Psyllium husk.

Likewise, Psyllium husk can be integrated with other ingredients into compound form. For example, 500 milligrams of Psyllium husk can be combined with 60 milligrams of Olive leaf; 40 milligrams of Hawthorne berry and 400 milligrams of parsley to produce a compound to be taken conveniently. Different amounts of each ingredient can be used. Also different ingredients can be used.

In general, Psyllium husk acts as a natural fiber to expand and absorb. Ingested as a fiber, Psyllium husk works to remove iron and additional toxins. Larger dosages increase the ability to draw out toxins. It is assumed that reducing fiber content of Psyllium husk (e.g., by transforming to a liquid) would reduce the ability to draw out toxins. Also, when combining Psyllium husk with other ingredients, care must be taken to avoid combining with ingredients that work against the ability of Psyllium husk to draw out iron and other toxins. A pure form of Psyllium husk can be a beneficial form to take.

For example, a method for lowering iron in blood might include the following (1) checking iron level in blood; (2) taking daily an amount of Psyllium husk predetermined to be effective in reducing iron level in blood; (3) monitoring iron level; and (4) ceasing or significantly reducing daily intake of Psyllium husk when iron level in blood is detected to be at a desired level. For example, the amount of Psyllium is 1500 milligram taken in 500 milligram doses spaced throughout each day. For example, the method may additionally include avoiding or minimizing intake of iron. For example, the method may additionally include at least one of the following: (1) drinking lots of fluids; (2) eating only foods that are low in iron are low in iron or have no iron; (3) avoiding blood transfusions; (4) foregoing intake of iron supplements; and (5) consuming three meals per day.

While the above, was discussed lowering iron in blood, Psyllium husk can be used to reduce other levels. For example, Psyllium husk can be used to reduce levels of, for example, lead, copper and other metals and minerals.

The foregoing discussion discloses and describes merely exemplary methods and embodiments. As will be understood by those familiar with the art, the disclosed subject matter may be embodied in other specific forms without departing from the spirit or characteristics thereof. Accordingly, the present disclosure is intended to be illustrative, but not limiting, of the scope of the invention, which is set forth in the following claims.

What is claimed is:

1. A method for lowering iron in blood, comprising:
checking iron level in blood,
taking daily an amount of Psyllium husk predetermined to be effective in reducing iron level in blood;
monitoring iron level; and,
ceasing or significantly reducing daily intake of Psyllium husk when iron level in blood is detected to be at a desired level.

2. A method as claim 1 wherein the amount of Psyllium is 1500 milligram taken in 500 milligram doses spaced throughout each day.

3. A method as in claim 1 additionally comprises,
avoiding or minimizing intake of iron.

4. A method as in claim 1 additionally comprising at least one of the following:
drinking lots of fluids;
eating only foods that are low in iron are low in iron or have no iron
avoiding blood transfusions;
foregoing intake of iron supplements; and,
consuming three meals per day.

5. A pill used for reducing iron in blood comprising:
Psyllium husk;
olive leaf;
Hawthorne berry; and,
Parsley.

6. A pill as in claim 5 wherein ingredient amounts are as follows:
Psyllium husk: 500 milligrams;
olive leaf: 60 milligrams;
Hawthorne berry: 40 milligrams; and,
Parsley: 400 milligrams.

7. A compound used for reducing iron in blood comprising:
Psyllium husk;
olive leaf;
Hawthorne berry; and,
Parsley.

8. A compound as in claim 5 wherein ingredient amounts are as follows:
Psyllium husk: 500 milligrams;
olive leaf: 60 milligrams;
Hawthorne berry: 40 milligrams; and,
Parsley: 400 milligrams.

9. A method for lowering targeted metals and minerals in blood, comprising:
checking a level of a targeted metal or mineral in blood,
taking daily an amount of Psyllium husk predetermined to be effective in reducing the level of the targeted metal or mineral in blood;
monitoring the level of the targeted metal or mineral; and,
ceasing or significantly reducing daily intake of Psyllium husk when the level of the targeted metal or mineral in blood is detected to be at a desired level.

10. A method as claim 9 wherein the amount of Psyllium is 1500 milligram taken in 500 milligram doses spaced throughout each day.

11. A method as in claim 9 additionally comprises,
avoiding or minimizing intake of iron.

12. A method as in claim 9 additionally comprising at least one of the following:
drinking lots of fluids;
eating only foods that are low in iron are low in iron or have no iron
avoiding blood transfusions;
foregoing intake of iron supplements; and,
consuming three meals per day.

13. A method as in claim 9 wherein the targeted metal or mineral is one of the following:
lead;
copper.
